(19) United States
(12) Patent Application Publication
(10) Pub. No.: US 2014/0163445 A1
(43) Pub. Date: Jun. 12, 2014

(54) LATTICE STRUCTURE MADE BY ADDITIVE MANUFACTURING

(71) Applicants: Jari Heikki Petteri Pallari, Rovaniemi (FI); Mikael Lars Justinus De Bruijn, Den Haag (NL)

(72) Inventors: Jari Heikki Petteri Pallari, Rovaniemi (FI); Mikael Lars Justinus De Bruijn, Den Haag (NL)

(73) Assignee: MATERIALISE NV, Leuven (BE)

(21) Appl. No.: 14/236,088

(22) PCT Filed: Aug. 3, 2012
(86) PCT No.: PCT/EP2012/065206

§ 371 Date: Jan. 30, 2014

(30) Foreign Application Priority Data
Aug. 5, 2011 (GB) 1113506.8

(51) Int. Cl. A61K 8/02; A61F 13/12; A61M 35/00

(52) U.S. Cl. CPC A61K 8/02; A61M 35/00; A61F 13/122
USPC 602/43; 604/290; 2/455; 132/200; 428/131; 29/460

(57) ABSTRACT

The present invention relates to free-form structures made by additive manufacturing, and methods for the manufacture thereof. The rigid free-form structures comprise a lattice structure which is impregnated by a polymeric or other material. The rigid free-form structures may be used in wound treatment, e.g. as a facial mask.

### FIELD OF THE INVENTION

The present invention relates to free-form structures made by additive manufacturing, and methods for the manufacture thereof. The free-form structures find use in wound treatment, compression therapy, orthotics and prosthetics.

### BACKGROUND

Scars are areas of fibrous tissue that replace normal skin after injury. Specifically, scar tissue is formed following an injury by connective tissue (non-elastic collagen fibers) that replaces normal soft functional tissue. Thus, scarring is a natural part of the healing process. However, scars may cause functional problems and affect a patient's self-esteem, particularly with burn wounds. Particular challenges are associated with the treatment of facial burns. Because of their proximity to the eyes, nose, ears and nasal passages, facial burns can present serious visual and pulmonary complications. It is known to use a custom burn mask to promote the healing process and minimize scarring. A custom burn mask is a clear plastic orthosis designed from a model of the patient's face and fit against the skin. The mask contacts the skin directly, or via a liner which is typically made from silicone or other polymeric material. The mask is used to apply direct pressure over the wound site to help prevent significant build up of collagen fibers and realign them in more normal formations. This will reduce the chance of hypertrophic scarring. The mask further protects the wound site from unwanted shear forces that could impair the healing process, and provide a barrier from potential irritants.

The facial masks known in the art typically consist of a shell with a constant thickness, as such masks are easier to manufacture. However, the uniform thickness means that the stiffness of different areas of the mask is not well controlled. This can result in the application of wrong amounts of pressure on certain regions of the wound site. While additive manufacturing methods in principle allow the fabrication of all sorts of structures, these methods currently do not allow the use of materials which are sufficiently durable and transparent for application in facial masks. Furthermore, the state of the art manufacturing method can result in a bad fit in approximately one third of the cases, which causes discomfort for the patient and/or reduces the effectiveness of the mask. A mask should be worn up to 18 hours per day and a good fit is essential for it to be comfortable and to stay in position.

Accordingly, there is a need for improved custom facial masks and other free-form structures which mitigate at least one of the problems stated above.

### SUMMARY OF THE INVENTION

The present invention relates to free-form structures fitting the surface of a body part. In particular embodiments, the free-form structures according to the present invention are custom facial masks, although the invention is not limited to such applications.

The present invention provides free-form structures fitting the surface of a body part, which are at least partially made by additive manufacturing. The free-form structures comprise a basis structure comprising a rigid lattice structure and a coating material provided thereon. In particular embodiments the lattice structure is impregnated in and/or enclosed by a coating material which is selected from a polymeric material, a ceramic material and/or a metal. In certain embodiments, the polymer is chosen from silicone, polyurethane, polyepoxide, polyamides, or blends thereof. In particular embodiments, the lattice structure is impregnated in and/or enclosed by a foamed solid. In certain embodiments, the free-form structure comprises a polymer layer with varying thickness.

In certain embodiments, the lattice structure is defined by a plurality of unit cells with a size between 1 and 20 mm. In particular embodiments, lattice structure is provided with varying unit cell geometries, varying unit cell dimensions and/or varying structure densities. In particular embodiments, the lattice structure comprises at least two separate lattice structure parts movably connected to each other and integrated into said structure.

In certain embodiments, the free-form structure further comprises one or more external and/or internal sensors (e.g. pressure and/or temperature sensors).

In particular embodiments, the free-form structure is a wound dressing device. In certain embodiments, the free-form structure is a facial mask, an orthopedic device, a protective helmet, a prosthetic device or a prosthetic socket.

In addition methods are provided for manufacturing free-form structures. The methods according to the present invention comprise the steps of:
a) providing a three dimensional representation of a body part of a subject;
b) designing a free-form structure comprising a lattice structure matching at least part of the surface of said body part, based on said three dimensional representation;

The methods envisaged may further comprise the steps of:
c) manufacturing the designed free-form lattice structure by additive manufacturing; and
d) impregnating or covering at least part of the free-form lattice structure with a polymer.

In further embodiments, step d) in the method according to the present invention is an overmolding process.

Also provided is the use of a free-form structure according to the present invention as a facial mask, preferably a burn mask. Also provided is the use of a free-form structure as described herein for the delivery of treatment agents to the skin. The use of a free-form structure as described herein for cosmetic purposes is also provided.

The application further provides a free-from composite material comprising a rigid lattice structure made by additive manufacturing said rigid lattice structure being at least partly covered, impregnated in and/or enclosed by a polymeric material, for use in wound treatment.

The free-form structures according to the present invention can have a different stiffness in different parts of the structure and can be made transparent, even though they are made at least partially via additive manufacturing. The free-form structures according to the present invention can further be made as a single part, and may further comprise internal or external sensors.

### BRIEF DESCRIPTION OF THE DRAWINGS

The following description of the figures of specific embodiments of the invention is merely exemplary in nature and is not intended to limit the present teachings, their application or uses. Throughout the drawings, corresponding reference numerals indicate like or corresponding parts and features.

FIG. 1 Illustration of a free-form structure comprising a rigid lattice structure (1) and coating (5) according to a particular embodiment of the present invention, for use as a facial mask.

FIG. 2 Basic structure comprising a lattice structure (2), bottom half (3) and top half of the mould (3′) for use in the overmolding process according to a particular embodiment of the present invention.

FIG. 3 Exploded view of suitable lattice structures according to particular embodiments of the present invention (Wadley H N, Phil. Trans. R. Soc. A 2006; 364:31-68).

In the figures, the following numbering is used:
1—free-form structure
2—rigid lattice structure
3, 3′—mould
4—unit cell
5—polymeric material
6—hole

### DETAILED DESCRIPTION

The present invention will be described with respect to particular embodiments but the invention is not limited thereto but only by the claims. Any reference signs in the claims shall not be construed as limiting the scope thereof.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” when referring to recited members, elements or method steps also include embodiments which “consist of” said recited members, elements or method steps.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

The recitation of numerical ranges by endpoints includes all numbers and fractions subsumed within the respective ranges, as well as the recited endpoints.

All documents cited in the present specification are hereby incorporated by reference in their entirety.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Free-form structures are provided herein, which fit at least part of the surface, i.e. external contour of a body part. The free-form structure is at least partially made by additive manufacturing and comprises a basic structure comprising or consisting of a lattice structure. The lattice structure may have one or more of the following advantages; the lattice structure may ensure and/or contribute to the fact that free-form structure has a defined rigidity and the lattice structure may ensure optimal coverage by the coating. In particular embodiments the lattice structure can contribute to the transparency of the structure. In particular embodiments of the free-form structures envisaged, a coating material is provided on the lattice structure. In particular embodiments, the lattice structure is at least partly covered by, impregnated in and/or enclosed by the coating material. This will be explained in more detail herein below.

The present invention provides a free-form structure. The term “free-form structure” as used herein refers to a structure having an irregular and/or asymmetrical flowing shape or contour, more particularly fitting at least part of the contour of one or more body parts. Thus, in particular embodiments, the free-form structure is a free-form surface. A free-form surface refers to an (essentially) two-dimensional shape contained in a three-dimensional geometric space. Indeed, as will be detailed below, such a surface can be considered as essentially two-dimensional in that it has limited thickness, but may nevertheless to some degree have a varying thickness. As it comprises a lattice structure rigidly set to mimic a certain shape it forms a three-dimensional structure. Typically, the free-form structure or surface is characterized by a lack of corresponding radial dimensions, unlike regular surfaces such as planes, cylinders and conic surfaces. Free-form surfaces are known to the skilled person and widely used in engineering design disciplines. Typically non-uniform rational B-spline (NURBS) mathematics is used to describe the surface forms; however, there are other methods such as Gorden surfaces or Coons surfaces. The form of the free-form surfaces are characterized and defined not in terms of polynomial equations, but by their poles, degree, and number of patches (segments with spline curves). Free-form surfaces can also be defined as triangulated surfaces, where triangles are used to approximate the 3D surface. Triangulated surfaces are used in STL (Standard Triangulation Language) files which are known to a person skilled in CAD design. The free-form structures according to the present invention fit the surface of a body part, as a result of the presence of a rigid basic structures therein, which provide the structures their free-form characteristics.

The term “rigid” when referring to the lattice structure and/or free-form structures comprising them herein refers to a structure showing a limited degree of flexibility, more particularly, the rigidity ensures that the structure forms and retains a predefined shape in a three-dimensional space prior to, during and after use and that this overall shape is mechanically and/or physically resistant to pressure applied thereto. In particular embodiments the structure is not foldable upon itself without substantially losing its mechanical integrity, either manually or mechanically. Despite the overall rigidity of the shape of the envisaged structures, the specific stiffness of the structures may be determined by the structure and/or material of the lattice structure. Indeed, it is envisaged that the lattice structures and/or free-form structures, while maintaining their overall shape in a three-dimensional space, may have some (local) flexibility for handling. As will be detailed below (local) variations can be ensued by the nature of the pattern of the lattice structure, the thickness of the lattice structure and the nature of the material. Moreover, as will be detailed below, where the free-form structures envisaged herein comprise separate parts (e.g. non-continuous lattice structures) which are interconnected (e.g, by hinges or by areas of coating material), the rigidity of the shape may be limited to each of the areas comprising a lattice structure.

The free-form structure according to the present invention comprises at least one rigid lattice structure, i.e. a structure which consists of an open framework, for example made of strips, bars, girders, beams or the like, which are contacting, crossing or overlapping in a regular pattern. The strips, bars, girders, beams or the like may have a straight shape, but may also have a curved shape. The lattice is not necessarily made of longitudinal beams or the like, and may for example consist of interconnected spheres, pyramids, etc.

Thus, the lattice structure is typically a framework which contains a regular, repeating pattern, wherein the pattern can be defined by a certain unit cell. A unit cell is the simplest repeat unit of the pattern. Thus, the lattice structure is defined by a plurality of unit cells. The unit cell shape may depend on the required stiffness and can for example be triclinic, monoclinic, orthorhombic, tetragonal, rhombohedral, hexagonal or cubic. Typically, the unit cells of the lattice structures have a volume ranging from 1 to 8000 mm³, preferably from 8 to 3375 mm³, more preferably from 64 to 3375 mm³, most preferably from 64 to 1728 mm³. The unit cell size determines, among with other factors such as material choice and unit cell geometry, the rigidity (stiffness) and transparency of the free-form structure. Larger unit cells generally decrease rigidity and increase transparency, while smaller unit cells typically increase rigidity and decrease transparency. Local variations in the unit cell geometry and/or unit cell size may occur, in order to provide regions with a certain stiffness (see further). Therefore, the lattice may comprise one or more repeated unit cells and one or more unique unit cells.

In order to ensure the stability of the lattice structure, the strips, bars, girders, beams or the like preferably have a thickness or diameter of 0.1 mm or more. In particular embodiments, the strips, bars, girders, beams or the like preferably have a thickness or diameter of 0.2 mm, 0.4 mm, 0.6 mm, 0.8 mm, 1 mm, 1.5 mm, 2 mm, 3 mm, 5 mm or more. The main function of the lattice structure is to ensure a certain stiffness of the free-form structure. The lattice structure may further enhance or ensure transparency, as it is an open framework. The lattice structure can preferably be considered as a reticulated structure having the form and/or appearance of a net or grid.

The stiffness of the lattice structure depends on factors such as the structure density, which depends on the unit cell geometry, the unit cell dimensions and the dimensions of the strips, bars, girders, beams, etc. of the framework. An important factor is the distance between the strips and the like, or in other words, the dimensions of the openings in the lattice structure. Indeed, the lattice structure is an open framework and therefore comprises openings. In particular embodiments, the opening size of the lattice structure is between 1 and 20 mm, between 2 and 15 mm or between 4 and 15 mm. In preferred embodiments, the opening size is between 4 and 12 mm. The size of the openings may be the equal to or smaller than the size of the unit cell.

In particular embodiments, the free-form structures of the present invention comprise a lattice structure comprising one or more interconnected reticulated layers. Preferably the lattice structure comprises one, two, three or more reticulated layers, i.e. the structure comprises different at least partially superimposed and interconnected layers within the lattice structure. The degree of stiffness provided by the lattice structure increases with the number of reticulated layers provided therein. In further particular embodiments, as detailed below, the free-form structures of the present invention comprise more than one lattice structure.

For certain applications the lattice structure may further comprise one or more holes with a larger size than the openings or unit cells as described herein above. Additionally or alternatively, the lattice structure does not extend over the entire shape of the free-form structure, such that openings in the structure, regions for handling (tabs or ridges) and/or regions of unsupported coating material are formed. An example of such an application is a facial mask, wherein holes are provided at the location of the eyes, mouth and/or nose holes. Typically, these latter holes are also not filled by the coating material. Accordingly, in particular embodiments, the size of the openings which are impregnated in and/or enclosed by the adjoining material ranges between 1 and 20 mm. The holes in the lattice structure (corresponding to holes in the free-form structure) as described herein above will also typically have a size which is larger than the unit cell. Accordingly, in particular embodiments, the unit cell size ranges between 1 and 20 mm.

According to particular embodiments the envisaged free-form structure contains regions comprising only the coating material is present. This may be of interest in areas where extreme flexibility of the free-form structure is desired.

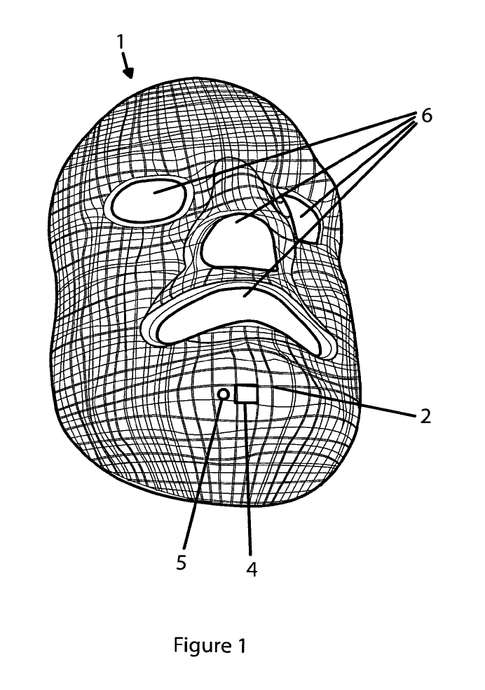

In particular embodiments, the envisaged free-form structure comprises a basic structure which contains, in addition to a lattice structure, one or more limited regions which do not contain a lattice structure, but are uniform surfaces. Typically these form extensions from the lattice structure with a symmetrical shape (e.g. rectangular, semi-circle). Such regions however typically encompass less than 50%, more particularly less than 30%, most particularly less than 20% of the complete basic structure. Typically they are used as areas for handling (manual tabs) of the structure and/or for placement of attachment structures (clips, elastic string etc.) In particular embodiments, the basic structure consist essentially only of a lattice structure (e.g. as illustrated in FIG. 1).

The facial masks known in the art typically have a uniform thickness. The average stiffness of such masks typically is controlled via the thickness and the choice of material, such that there is no difference in the stiffness of different regions of the mask relative to each other. However, in many cases, it can be advantageous for the structure to have certain regions with a different stiffness. In the present invention, this can be achieved by providing a lattice structure with locally varying unit cell geometries, varying unit cell dimensions and/or varying densities and/or varying thicknesses of the lattice structure (by increasing the number of reticulated layers). Accordingly, in particular embodiments, the lattice structure is provided with varying unit cell geometries, varying unit cell dimensions, varying lattice structure thicknesses and/or varying densities. Additionally or alternatively, as will be detailed below, the thickness of the coating material may also be varied. Thus, in particular embodiments, the free-form structure has a varying thickness. In further particular embodiments, the free-form structures according to the present invention have regions with a different stiffness, while they retain the same volume and external dimensions.

The free-form structure according to the present invention is at least partially made by additive manufacturing (AM). More particularly it is envisaged that at least the basic structure comprising the lattice structure comprised by the free-form structure according to the present invention is made by additive manufacturing. Additive Manufacturing can be defined as a group of techniques used to fabricate a tangible model of an object typically using three-dimensional (3-D) computer aided design (CAD) data of the object. Currently, a multitude of Additive Manufacturing techniques is available, including stereolithography, Selective Laser Sintering, Fused Deposition Modeling, foil-based techniques, etc.

Selective laser sintering uses a high power laser or another focused heat source to sinter or weld small particles of plastic, metal, or ceramic powders into a mass representing the 3-dimensional object to be formed.

Fused deposition modeling and related techniques make use of a temporary transition from a solid material to a liquid state, usually due to heating. The material is driven through an extrusion nozzle in a controlled way and deposited in the required place as described among others in U.S. Pat. No. 5,141,680.

Foil-based techniques fix coats to one another by means of gluing or photo polymerization or other techniques and cut the object from these coats or polymerize the object. Such a technique is described in U.S. Pat. No. 5,192,539.

Typically AM techniques start from a digital representation of the 3-D object to be formed. Generally, the digital representation is sliced into a series of cross-sectional layers which can be overlaid to form the object as a whole. The AM apparatus uses this data for building the object on a layer-by-layer basis. The cross-sectional data representing the layer data of the 3-D object may be generated using a computer system and computer aided design and manufacturing (CAD/CAM) software.

The basic structure comprising or consisting of the lattice structure may thus be made of any material which is compatible with additive manufacturing and which is able to provide a sufficient stiffness to the rigid shape of the regions comprising the lattice structure in the free-form structure or the free-form structure as a whole. Suitable materials include, but are not limited to polyurethane, acrylonitrile butadiene styrene (ABS), polycarbonate (PC), PC-ABS, polyamide, polyamide with additives such as glass or metal particles, methyl methacrylate-acrylonitrile-butadiene-styrene copolymer, etc. Examples of commercially available materials are: DSM Somos® series of materials 7100, 8100, 9100, 9420, 10100, 11100, 12110, 14120 and 15100 from DSM Somos; ABSplus-P430, ABSi, ABS-ESD7, ABS-M30, ABS-M30i, PC-ABS, PC-ISO, PC, ULTEM 9085, PPSF and PPSU materials from Stratasys; Accura Plastic, DuraForm, CastForm, Laserform and VisiJet line of materials from 3-Systems; Aluminium, CobaltChrome and Stainless Steel materials; Maranging Steel; Nickel Alloy; Titanium; the PA line of materials, PrimeCast and PrimePart materials and Alumide and CarbonMide from EOS GmbH.

In particular embodiments of the free-form structures of the present invention, the basic structure or the lattice structure comprised therein as described herein above is covered at least in part with a coating material, preferably a material which is different from the material used for manufacturing the lattice structure. In particular embodiments the lattice structure is at least partly embedded within or enclosed by (and optionally impregnated with) the coating material. In further embodiments, the coating material is provided onto one or both surfaces of the lattice structure. In particular embodiments only certain surface regions of the basic structure and/or the lattice structure in the free-form structure are provided with a coating material. In particular embodiments, at least one surface of the basic structure and/or lattice structure is coated for at least 50%, more particularly at least 80%. In further embodiments, all regions of the basic structure having a lattice structure are fully coated, on at least one side, with the coating material. In further particular embodiments, the basic structure is completely embedded with the coating material, with the exceptions of the tabs provided for handling.

In further embodiments, the free-form structure comprises, in addition to a coated lattice structure, regions of coating material not supported by a basic structure and/or a lattice structure.

Accordingly, in particular embodiments, the free-form structure of the present invention comprises at least two materials with different texture or composition. In preferred embodiments, the free-form structure according to the present invention is a composite structure, i.e. a structure which is made up of at least two distinct compositions and/or materials.

The coating material(s) may be a polymeric material, a ceramic material and/or a metal. In particular embodiments, the coating material(s) is a polymeric material. Suitable polymers include, but are not limited to, silicones, a natural or synthetic rubber or latex, polyvinylchloride, polyethylene, polypropylene, polyurethanes, polystyrene, polyamides, polyesters, polyepoxides, aramides, polyethyleneterephthalate, polymethylmethacrylate, ethylene vinyl acetate or blends thereof. In particular embodiments, the polymeric material comprises silicone, polyurethane, polyepoxide, polyamides, or blends thereof.

In particular embodiments the free-form structures comprise more than one coating material or combinations of different coating materials.

In specific embodiments, the coating material is a silicone. Silicones are typically inert, which facilitates cleaning of the free-form structure.

In particular embodiments, the coating material is an optically transparent polymeric material. The term “optically transparent” as used herein means that a layer of this material with a thickness of 5 mm can be seen through based upon unaided, visual inspection. Preferably, such a layer has the property of transmitting at least 70% of the incident visible light (electromagnetic radiation with a wavelength between 400 and 760 nm) without diffusing it. The transmission of visible light, and therefore the transparency, can be measured using a UV-Vis Spectrophotometer as known to the person skilled in the art. Transparent materials are especially useful when the free-form structure is used for wound treatment (see further). The polymers may be derived from one type of monomer, oligomer or prepolymer and optionally other additives, or may be derived from a mixture of monomers, oligomers, prepolymers and optionally other additives. The optional additives may comprise a blowing agent and/or one or more compounds capable of generating a blowing agent. Blowing agents are typically used for the production of a foam.

Accordingly, in particular embodiments, the coating material(s) are present in the free-form structure in the form of a foam, preferably a foamed solid. Thus, in particular embodiments, the lattice structure is coated with a foamed solid. Foamed materials have certain advantages over solid materials: foamed materials have a lower density, require less material, and have better insulating properties than solid materials. Foamed solids are also excellent impact energy absorbing materials and are therefore especially useful for the manufacture of free-form structures which are protective elements (see further). The foamed solid may comprise a polymeric material, a ceramic material or a metal. Preferably, the foamed solid comprises one or more polymeric materials.

The foams may be open cell structured foams (also known as reticulated foams) or closed cell foams. Open cell structured foams contain pores that are connected to each other and form an interconnected network which is relatively soft. Closed cell foams do not have interconnected pores and are generally denser and stronger than open cell structured foams. In particular embodiments, the foam is an “Integral skin foam”, also known as “self-skin foam”, i.e. a type of foam with a high-density skin and a low-density core.

Thus in particular embodiments, free-form structures comprise a basic structurewhich comprise a lattice structure which is at least partially coated by a polymeric or other material as described herein above. For some applications, the thickness of the coating layer and the uniformity of the layer thickness of the coating are not essential. However, for certain applications, it can be useful to provide a layer of coating material with an adjusted layer thickness in one or more locations of the free-form structure, for example to increase the flexibility of the fit of the free-form structure on the body part. An example of such an application is a compression glove for the treatment of burn wounds. In such a glove, areas around the joints may be provided with a thinner layer of coating material to enhance the mobility of the fingers. Accordingly, in particular embodiments, the free-form structure according to the present invention is provided with a varying thickness of the coating layer.

The shape of the free-form structure according to the present invention is typically complementary to the surface of one or more body parts. This in order to allow positioning of the free-form structure on said body part of a person or animal, whereby equal pressure is applied over substantially the whole area of the body part covered by the structure. This can help prevent significant build up of collagen fibers and help realign them in the desired formation.

The body part(s) for which the structures described herein may be designed may be any part of the human or animal body, for example the head, the face, a leg (or part thereof), an arm (or part thereof), a hand (or part thereof), etc. The specific body part(s) depend on the specific envisaged function of the free-form structure.

In particular embodiments, the free-form structure according to the present invention is used as a wound dressing device. For example, in particular embodiments, the free-form structure can be used to promote healing and minimize scarring, by providing a uniform pressure to a wound and optionally by ensuring the delivery of treatment agents to the skin. In further embodiments, the free-form structure is used for dressing a burn wound. The fact that the free-form structure can be made transparent is particularly useful for wound treatment. This way, the free-form structure can protect the wound, while still allowing visual inspection of the wound site by a physician, e.g. to see changes in surface circulation. In specific embodiments, the free-form structure is a facial mask, for example for the treatment of facial (burn) wounds. In further embodiments, the free-form structure is a burn mask.

In particular embodiments, the free-form structure according to the present invention is used as a cosmetic device. In specific embodiments, the free-form structure is a facial mask, for example a beauty mask. The cosmetic devices may provide a uniform pressure to the facial skin, and/or may be used for the delivery of treatment agents to the skin. The treatment agents may be applied to the free-form structure surface, may be contained by an open cell structured foam as described hereabove, or may be delivered via channels (see further). The cosmetic devices may further also be capable of heating or cooling the skin, for example via the circulation of fluids in internal channels (see further).

In particular embodiments, the free-form structure is used as a protective element, which protects one or more body parts. In certain embodiments, the free-form structure is a protective helmet.

In particular embodiments, the free-form structure is used as an orthopedic device, which includes devices that can be used to treat or repair defective, diseased, or damaged tissue of the muscular/skeletal system(s). In certain embodiments, the free-form structure is a prosthetic device, for example for hands, feet, fingers, or a component of a prosthetic device, for example a socket for a prosthetic device.

Accordingly, in certain embodiments, the free-form structure is used or designed for use as a facial mask, an orthopedic device or a protective helmet.

The basic structure of the freeform structures envisaged herein can be made as a single rigid free-form part which does not need a separate liner or other elements. Independent thereof it is envisaged that the free-form structures according to the present invention can be further provided with additional components such as sensors, straps or other means for maintaining the structure in position on the body, or any other feature that may be of interest in the context of the use of the structures of the invention.

In certain embodiments, the free-form structure comprises a single rigid lattice structure (optionally comprising different interconnected layers of reticulated material). However, such structures often only allow a limited flexibility, which may cause discomfort to a person or animal wearing the free-form structure. A significant increase in flexibility can be obtained if the free-form structure comprises two or more separate rigid lattice structures which can move relative to each other. These two or more lattice structures are then enclosed by a material as described above, such that the resulting free-form structure still is made or provided as a single part. The rigidity of the shape of the free-form structure is ensured locally by each of the lattice structures, while additional flexibility during placement is ensured by the fact that there is a (limited) movement of the lattice structures relative to each other. Indeed, in these embodiments, the coating material and/or a more limited lattice structure) will typically ensure that the lattice structures remain attached to each other.

In particular embodiments, the lattice structures are partially or completely overlapping. However, in particular embodiments, the different lattice structures are non-overlapping. In further particular embodiments, the lattice structures are movably connected to each other, for example via a hinge. In particular embodiments the connection is ensured by lattice material. In further particular embodiments the lattice structures may be interconnected by one or more beams which form extensions of the lattice structures. In further particular embodiments the lattice structures are held together in the free-form structure by the coating material. An example of such a free-form structure is a facial mask with a jaw structure that is movable with respect to the rest of the mask. Accordingly, in particular embodiments, the lattice structure comprises at least two separate lattice structures movably connected to each other, whereby the lattice structures are integrated into the free-form structure.

The free-form structure according to the present invention may be used for wound treatment as described hereabove. For optimal healing, it is important that the free-form structure provides a uniform contact and/or pressure on the wound site or specific locations of the wound site. With conventional devices for wound treatment such as masks, pressure monitoring is not straightforward. The lattice structure makes it simple to incorporate pressure sensors into the free-form structure according to the present invention. The sensors can be external sensors, but may also be internal sensors. Indeed, the lattice structure can be designed such that it allows mounting various sensors at precise locations, before impregnating and/or enclosing the lattice structure by a polymer or other material.

Additionally or alternatively, the free-form structure may comprise one or more other sensors such as a temperature sensor, a moisture sensor, an optical sensor, a strain gauge, an accelerometer, a gyroscope, a GPS sensor, a step counter, etc. Accelerometers, gyroscopes, GPS sensors and/or step counter may for example be used as an activity monitor. Temperature sensor(s), moisture sensor(s), strain gauge(s) and/or optical sensor(s) may be used to monitor the healing process during wound treatment. Specifically, the optical sensor(s) may be used to determine collagen fiber structure as explained in US patent application 2011/0015591, which is hereby incorporated by reference.

Accordingly, in particular embodiments the free-form structure further comprises one or more external and/or internal sensors. In specific embodiments, the free-form structure comprises one or more internal sensors. In certain embodiments, the free-form structure comprises one or more pressure and/or temperature sensors.

The skilled person will understand that in addition to the sensor(s), also associated power sources and/or means for transmitting signals from the sensor(s) to a receiving device may be incorporated into the free-form structure, such as wiring, radio transmitters, infrared transmitters, and the like.

In particular embodiments, at least one sensor comprises “Micro Electronic Mechanical Systems” (MEMS) technology, i.e. technology which integrates mechanical systems and micro-electronics. Sensors based on MEMS technology are also referred to as “MEMS-sensors”. Such sensors are small and light, and consume relatively little power. Non-limiting examples of suitable MEMS-sensors are the STTS751 temperature sensor and the LIS302DL accelerometer STMicroelectronics.

The lattice structure also allows providing the free-form structure with one or more (internal) channels. These channels may be used for the delivery of treatment agents to the skin. The channels may also be used for the circulation of fluids, such as heating or cooling fluids. Accordingly, in particular embodiments, the free-form structure according to the present invention is provided with one or more (internal) channels.

Additionally provided herein are methods for manufacturing a free-form structure, particularly a free-form structure as described herein above. In particular embodiments the free-form structures of the present invention are manufactured by providing a rigid free-form basic structure comprising a lattice structure based on a three dimensional representation of a body part using additive manufacturing and providing a coating material on said lattice structure so as to obtain the free-form structure.

In particular embodiments, the free-form structures of the present invention are patient-specific, i.e. they are made to fit specifically on the anatomy of a certain (animal or human) body-part. Accordingly, in particular embodiments, the method for manufacturing a free-form structure, particularly a free-form structure as described herein above comprises the steps of:
a) providing a three dimensional representation of a body part of a subject. Typically this implies providing a three-dimensional image. For this purpose, the subject may be scanned using a 3D scanner, e.g. a hand-held laser scanner. The collected data can then be used to construct a digital, three dimensional model of the body part of said subject.
b) designing a free-form structure based on said three dimensional representation of said body part, such that the structure is essentially complementary to at least part of said body part and comprises or consists of a lattice structure. In the lattice structure, one or more types and/or sizes of unit cell may be selected, depending on the subject shape, the required stiffness of the free-form structure, etc. Different lattice structures may be designed within the free-form structure for fitting on different locations on the body part. The different lattice structures may be provided with a hinge so that they can be connected and/or, can be digitally blended together or connected by beams in the basic structure to form a single part.

In particular embodiments, the methods for the provision of free-form structures further comprise the step of.
c) manufacturing said designed free-form structure by additive manufacturing.

In further particular embodiments, it is envisaged that the methods comprise the step of:
d) providing a coating material on said basic structure which coating material is preferably a polymer. In particular embodiments, the material is a foamed solid.

These different steps need not be performed in the same location or by the same actors. Indeed typically, the design of the free-form structure, the manufacturing and the coating are ensured in different locations by different actors. Moreover, it is envisaged that additional steps may be performed between the steps recited above.

In particular embodiments, step d) of the methods envisaged herein is an overmolding process. The overmolding process is a process known to the skilled person and typically comprises the step of designing a mold of the area of the body part to be covered by the free-form structure, manufacturing said mold, and providing the (one or more) lattice structure(s) therein and providing the coating material in the mold so as to form the free-form structure. The overmolding process may for instance comprise the steps of designing a mold around the lattice structure, manufacturing said mold, placing said lattice structure inside said mold, filling said mold with polymer material, allowing the polymer to set around and impregnate said lattice structure and removing the mold, thereby providing a free-form composite structure. In preferred embodiments, the mold is manufactured via additive manufacturing. In particular embodiments, the coating material is a foam and the foam is provided onto said lattice structure.

More particularly, step (a) according to the method of the invention provides the starting material, i.e. the patient-specific images. The images can be provided by a technician or medical practitioner by scanning the subject or part thereof. Such images can then be used as or converted into a three-dimensional representation of the subject, or part thereof. Additional steps wherein the scanned image is manipulated and for instance cleaned up may be envisaged.

Step (b) of the methods envisaged herein provides in designing a free-form basic structure based on said three dimensional representation of said subject or part thereof, whereby said basic structure comprises or consists of a lattice structure. This step includes typical designing steps such as designing trimlines for the mask with openings for the eyes, mouth and nose and performing other designing manipulations where required. This step also includes steps required for designing the lattice structure, including for instance defining surfaces on the positive print of the mask that need different properties, different cell sizes and/or openings, generate the cells with the required geometry and pattern them as needed on the defined surfaces to cover said surfaces, and combine the separate cell patterns into a single solid part. It should be stressed that the requirements of the lattice structure would be clear to a skilled person while designing the lattice structure. The skilled person will therefore use data obtained from his own experience as well as data from numerical modeling systems, such as FE and/or CFD models.

Step (c) of the methods envisaged herein provides in manufacturing said designed free-form structure by additive manufacturing. Typically, the structure This step may further include manufacturing the mould parts and assembling the mould parts together with the lattice structure. The mould parts may be made by commonly known techniques, but they may also be manufactured by additive manufacturing.

Step (d) of the methods envisaged herein provides in coating or impregnating said free-form basic structure comprising said lattice structure with a certain material, preferably a polymer, thereby generating the free-form structure. This step may include steps as adding the polymeric material or other material into the mould, curing the material impregnating the lattice structure and disassembling the mould.

After manufacturing the free-form structure, the structure may go through a number of post-process steps including for instance cleaning up and finishing the free-form structure.

Further provides different applications of a rigid free-form structure as described herein. As detailed above, different applications are envisaged for the free-form structures described herein, such as but not limited to therapeutic, cosmetic and protective applications.

Accordingly, the use of the free-form structures described herein in the care and treatment of damaged skin surfaces, such as burn wounds is envisaged. In further embodiments, the use of the free-from structures described herein in the care, protection and treatment of undamaged skin surfaces is envisaged. According to additional particular embodiments the use of a free-form structure as described herein for cosmetic purposes is envisaged. In further embodiments, the use of a free-form structure as described herein for the delivery of treatment agents to the skin is envisaged. In particular embodiments, the structure further comprises one or more therapeutic compositions. The therapeutic composition may be embedded in the coating material.

In further embodiments, the use of the structures described herein as prosthetic devices is envisaged, i.e. for replacing a body part. In these embodiments it is envisaged that the free-forms structure is made to be identical to the missing body part.

In yet further embodiments, the use of the structures described herein is envisaged in methods for the prevention of damage to one or more body parts. Examples of such embodiments are the provision of such free-form structures as helmets or protective wear for other body parts. The methods described herein encompass positioning of a free-form structure according to the invention which has been adapted to conform with a body part, on said body part.

In particular embodiments, the free-form structure is used as a facial mask. The mask may be used for (burn) wound treatment, for cosmetic purposes, or other purposes.

A rigid free-form material is also provided for use in medicine, and preferably for use in wound treatment. The rigid free-form composite material comprises a basic structure comprising or consisting of a lattice structure. The material is made by additive manufacturing, or at least partially made by additive manufacturing, and has a rigid free-form. In particular embodiments, the basic structure comprising or consisting of a lattice structure is coated with, impregnated in and/or enclosed by a polymeric material.

The present invention will be illustrated by the following non-limiting embodiments.

### EXAMPLES

### a) Facial Mask

FIG. 1 shows a free-form structure (1) according to a particular embodiment of the present invention. The free-form structure fits the surface of a person's face. Specifically, the free-form structure is a facial mask which can be used in (burn) wound treatment. FIG. 1 shows that the free-form structure comprises a lattice structure (2) which is impregnated in a polymeric material (5). The open lattice structure and the transparency of the polymer ensure that the free-form structure is transparent. The lattice structure is defined by unit cells (4) which have a size between 1 and 20 mm. The lattice structure is provided with varying unit cell geometries and dimensions, in order to control the stiffness of the free-form structure. The lattice structure further comprises holes (6) which are not impregnated, for the eyes, nose and mouth.

### b) Overmouldinq Process

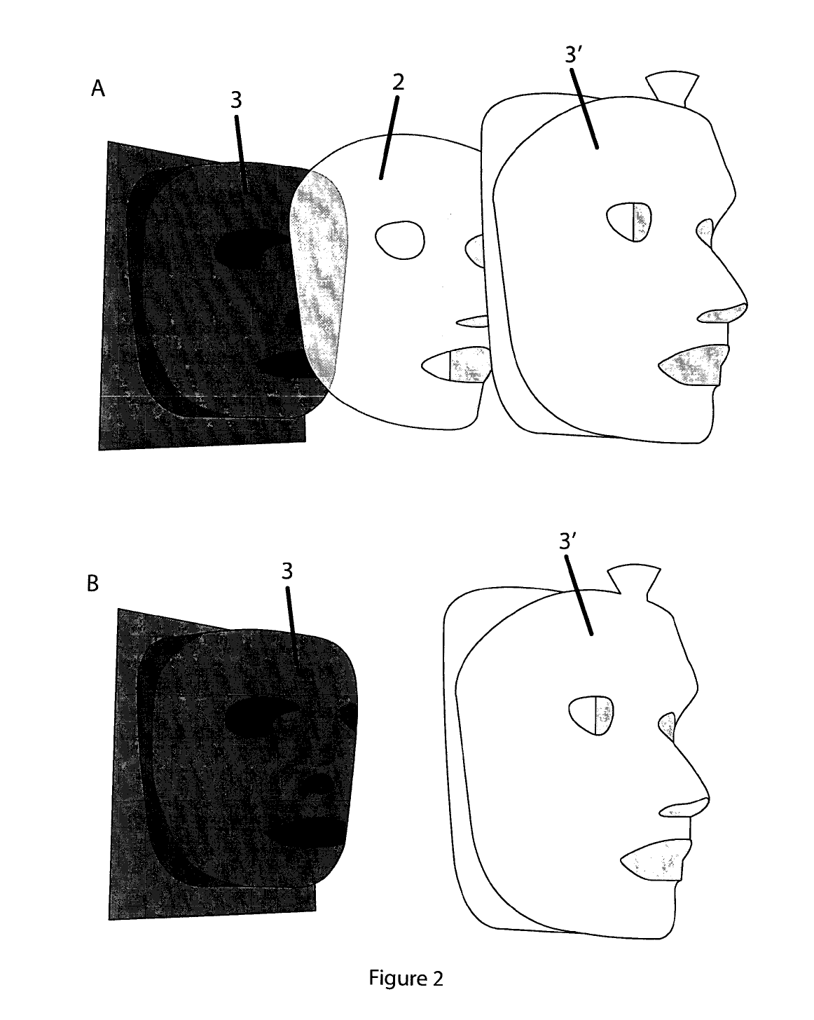

Methods for manufacturing a free-form structure are provided herein which involve the step of impregnating a basic structure comprising or consisting of a rigid lattice structure with a certain material, preferably a polymer. In particular embodiments, this is obtained via an overmoulding process. FIG. 2A shows two parts of an exemplary mould (3, 3′) and a lattice structure (2) which can be used in an overmoulding process for manufacturing a facial mask as shown in FIG. 1. In the overmoulding process, the lattice structure (2) is first placed in the mould (i.e. between the mould halves (3, 3′)). Then, the mould containing the lattice structure is filled with the impregnating (polymer) material. After setting of the impregnating material, the mould halves (3, 3′) are removed, yielding the coated structure.

### c) Lattice Structures

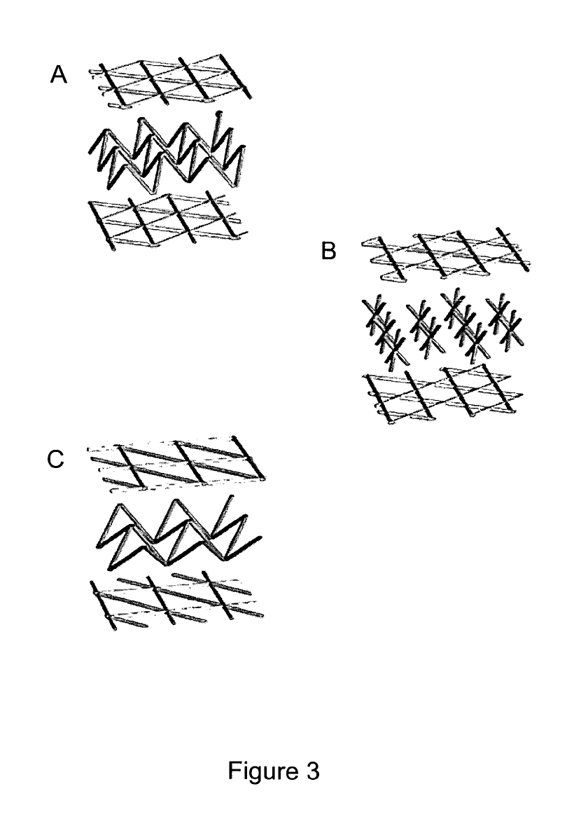

FIGS. 3 (A, B and C) shows exploded views of suitable lattice structures according to particular embodiments of the present invention. The lattice structures have open faces and are layered. The shown structures can be regarded as two interconnected reticulated layers, but also structures comprising only one layer, or more than two layers are envisaged.

1. A free-form structure fitting the surface of a body part, at least partially made by additive manufacturing, comprising a basic structure comprising or consisting of a rigid free-form lattice structure and a coating material provided thereon selected from the group comprising a polymeric material, a ceramic material and a metal.

2. The structure according to claim 1, wherein said coating material is a foamed solid.

3. The structure according to claim 1, wherein said coating material is a polymer such as silicone, polyurethane, polyepoxide, polyamides, or blends thereof.

4. The structure according to claim 1, wherein said rigid free-form lattice structure is defined by a plurality of unit cells with a size between 1 and 20 mm.

5. The structure according to claim 1, comprising at least two separate rigid free-form lattice structures movably connected to each other and integrated into said structure.

6. The structure according to claim 1, wherein said structure further comprises one or more external and/or internal sensors.

7. The structure according to claim 1, wherein said lattice structure has varying unit cell geometries, varying unit cell dimensions and/or varying structure densities.

8. The structure according to claim 1, wherein said coating material has a varying thickness.

9. The structure according to claim 1, wherein said body part is a face.

10. The structure according to claim 1, wherein said structure is an orthopedic device or a prosthetic device.

11. Method for manufacturing a free-form structure comprising the steps of:
a) providing a three dimensional representation of a body part of a subject;
b) designing a basic structure comprising a rigid free-form lattice structure based on said three dimensional representation of said subject;
c) manufacturing said designed basic structure by additive manufacturing; and; optionally
d) providing said basic structure with a coating material.

12. Method according to claim 11 wherein step d) is an overmolding process.

13-16. (canceled)

17. A method of treating a wound on a body part, comprising positioning onto said body part a free-form structure according to claim 1, fitting the surface of said body part.

18. A method for protecting a body part, comprising positioning onto said body part a free-form structure according to claim 1, fitting the surface of said body part.

19. A method for delivering a treatment agent to the skin of a body part, comprising:
(a) positioning onto said body part a free-form structure according to claim 1; and
(b) delivering said treatment agent to the skin of said body part via said free-form structure.

20. A method for cosmetic treatment of the skin of a body part, comprising positioning onto said body part a free-form structure according to claim 1, fitting the surface of said body part.
